(19) United States
(12) Patent Application Publication
(10) Pub. No.: US 2010/0317642 A1
(43) Pub. Date: Dec. 16, 2010

(54) PHARMACEUTICAL COMPOSITION OF ORLISTAT

(75) Inventors: H. C. Pandey, Hyderabad (IN); R. B. Radhakrishnan, Tamilnadu (IN); Sariha Farnaaz Mohammed, Hyderabad (IN); Dipali Vaghela, Ahmedabad (IN); Reddy Golamaru Lakshmi Narayana, Andhra Pradesh (IN)

Correspondence Address: MERCHANT & GOULD PC, P.O. BOX 2903, MINNEAPOLIS, MN, 55402-0903, US

(73) Assignee: INVENTIS DDS PVT LIMITED, Hyderabad (IN)

(21) Appl. No.: 12/738,145

(22) PCT Filed: Oct. 15, 2007
(86) PCT No.: PCT/IN07/00482

§ 371 Date: Aug. 5, 2010

(51) Int. Cl. A61K 31/397; A61P 3/04

(52) U.S. Cl. 514/210.02

(57) ABSTRACT

A stable pharmaceutical composition comprising dispersion blend comprising 20 to 60% by weight of orlistat and 40% to 80% by weight of water soluble polymer carrier selected from hydroxypropyl methyl cellulose, methylcellulose, hydroxyethyl cellulose, hydroxypropyl cellulose or hydroxypropyl methylcellulose and the like.

### FIELD OF INVENTION

The present invention relates to a stable composition comprising dispersion blend of orlistat and water soluble polymer. More particularly the present invention relates to a composition comprising a dispersion blend comprising orlistat with water soluble polymer in defined amount to act as stabilizer such that the composition is free flowing and has easy processability.

### BACKGROUND AND PRIOR ART

Obesity is a disease characterized by an excess body fat. A number of concomitant pathological processes and diseases are associated with obesity including coronary heart disease, hypertension, stroke, non-insulin dependent diabetes mellitus and certain forms of cancer. Obesity may be treated by surgery or pharmacological therapy, besides changes in diet, behaviour and physical activities. Appetite suppressants (amphetamine-like products) that act via the central nervous system (CNS) are the most commonly available medicinal products for treatment of obesity. However, they may not be prescribed for periods longer than three months, due to the potential risk of abuse. Thus, there has been a need for medicinal products that can be used in chronic treatment together with dietary and behavioural modifications.

Orlistat belongs to a new class of pharmacological agents. It inhibits the action of gastrointestinal lipases and thereby impairs the metabolism of lipids in the intestinal lumen leading to a prevention of lipid absorption.

Orlistat is indicated in conjunction with a mildly hypocaloric diet for the treatment of obese patients, or overweight patients with associated risk factors. The treatment should only be started if diet alone has previously produced a weight loss of at least 2.5 kg over a period of 4 consecutive weeks. Treatment with orlistat should be discontinued after 12 weeks if patients have been unable to lose at least 5% of the body weight as measured at the start of drug therapy. The recommended dose of orlistat is one 120 mg capsule three times daily, which should be taken immediately before, during or up to one hour after each main meal.

Orlistat, a hydrogenated derivative of lipstatin and an inhibitor of gastrointestinal lipases produced by chemical synthesis, is presently available as Xenical® 120 mg capsules.

The approved labeling of Xenical® describes it as a conventional hard gelatin capsule containing pellets with an active substance concentration of 50%. The excipients used include cellulose microcrystalline (as diluent and extrusion/spheronisation aid), sodium starch glycollate (as desintegrant), sodium lauryl sulphate (as wetting agent), povidone K30 (as binder and stabiliser), and talc is added (for lubrication) to the pellets before encapsulation.

Pharmaceutical formulations of olistat have been described in various patents and patent applications.

U.S. Pat. No. 6,004,996 describes product containing tetrahydrolipstatin or orlistat as the active ingredient and microcrystalline cellulose and polyvinylpyrrolidone as excipients, characterized in that it is in the form of particles with a diameter of 0.25 to 2 mm. The invention describes use of an extruder for preparation of these particles. U.S. Pat. No. 6,534,087, teaches a method for the preparation of compositions, preferably pharmaceutical compositions, in form of expanded, mechanically stable, lamellar, porous, sponge-like or foam structures out of solutions and dispersions results in a favored pharmaceutical product. This method comprises the steps of a) preparing a solution or a homogeneous dispersion of a liquid and a compound selected from the group consisting of one or more pharmaceutically active compounds, one or more pharmaceutically suitable excipients, and mixtures thereof, followed by b) the expansion of the solution or the homogeneous dispersion without boiling.

U.S. Pat. No. 6,358,522 provides orally administrable pharmaceutical compositions containing an inhibitor of gastrointestinal lipase, one (or more) additive(s) of the group consisting of substantially non-digestible, substantially non-fermentable, hydrophilic and/or hydrocolloidal food grade thickeners and emulsifiers, and auxiliary excipients. Methods are provided for preventing and treating anal leakage of oil in a patient by administering the compositions of the present invention to the patient.

U.S. Pat. No. 6,756,364 describes a pharmaceutical combination or composition containing a lipase inhibitor, preferably orlistat, and a bile acid sequestrant useful for treating obesity.

EP 638,317 also describes a pharmaceutical composition including orlistat.

However, there are no detailed disclosures regarding the production and purification of orlistat in the patent literature. Orlistat described in literature, sometimes, is not appropriate for preparing a robust pharmaceutical composition as it requires high purity. There is a continuous need to improve the preparation of orlistat.

Orlistat, a white to off-white crystalline powder, is a lipophilic substance with very low solubility in water within the physiological pH range. It is an inhibitor of pancreatic lipase. Due to its low melting point, of about 44° C., it undergoes both hydrolytic and thermal degradation, particularly when stored in a humid atmosphere or above 35° C. in a dry atmosphere. Furthermore, conventional dosage forms such as described in U.S. Pat. No. 4,598,089, for example, tablets or hard gelatin capsules, cannot easily be formulated from powder mix or by conventional wet granulation procedure due to picking and sticking phenomena during tablet compression or encapsulation. Thus, there was a need for orlistat containing products and dosage forms which would be stable against moisture and heat during production and storage.

U.S. Pat. No. 6,734,314 relates to the solid state physical properties of orlistat. These properties can be influenced by controlling the conditions under which orlistat is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

In prior art various techniques have been tried for using water soluble polymers for solubility enhancement. In a study (Mitchell S.A., et. al.; Int. J. of Parmaceutics, 250, 3 (2003)), three poorly water-soluble drugs, naproxen, nifedipine, and carbamazepine, were studied with low-viscosity HPMC USP Type 2208 (K3LV), HPMC USP Type 2910 (E3LV and E5LV), and methylcellulose. Polymer and drug were dry-blended, compressed into slugs on a tablet press or into ribbons on a roller compactor, and then milled into a granular powder. Dissolution testing of the milled powder was performed on USP Apparatus II, 100 rpm, 900 ml deionized water, 37 8 C. Drug distribution vs. particle size was also studied. The compaction processes enhanced drug dissolution relative to drug alone and also relative to corresponding loosely mixed physical mixtures. The roller compaction and slugging methods produced comparable dissolution enhancement. The mechanism for dissolution enhancement is believed to be a microenvironment HPMC surfactant effect facilitated by keeping the HPMC and drug particles in close proximity during drug dissolution. The compaction methods in this study may provide a lower cost, quicker, readily scalable alternative for formulating poorly water-soluble drugs.

In recent years, an increasing number of active agents possess low aqueous solubility. As a result, oral delivery of poorly water-soluble drugs often results in low bioavailability since the rate-limiting step for absorption from the gastrointestinal tract is a significantly slower dissolution rate.

A common approach to improve the dissolution rate of poorly water-soluble drugs, and, therefore, improve oral bioavailability is by formation of a solid dispersion (Chiou, W.L., Riegelman, S., J. Pharm. Sci. 60, 1281 (1971)); Serajuddin, A.T.M., J. Pharm. Sci. 88, 1058 (1999)) with a water-soluble rate-enhancing polymer, such as polyethylene glycol. Typical methods for fabricating solid dispersions include solution methods (Sumnu, M., STP Pharma 2, 214 (1986); Mura, P., Manderioli, A., Bramanti, G., Ceccarelli, L., Drug Dev. Ind. Pharm. 22, 909 (1996); Mura, P., Faucci, M.T., Manderioli, A., Bramanti, G., Parrini, P., Drug Dev. Ind. Pharm. 25, 257 (1999); Doshi, D.H., Ravis, W.R., Betageri, G.V., Drug Dev. Ind. Pharm. 23, 1167 (1997)) and melt methods (Yan, G., Li, H., Zhang, R., Ding, D., Drug Dev. Ind. Pharm. 26, 681 (20000)), but these techniques are not readily scalable and have the disadvantages of solvent use and potential drug degradation at elevated temperatures.

Broman etal (Broman, E., Khoo, C., Taylor, L.S., Int. J. Pharm. 222, 139 (2001).) describes a novel method to fabricate solid dispersions utilizing compression moulding, but this technique also utilizes elevated temperature and a thermoplastic polymer. Several researchers have developed methods to use hydroxypropyl methylcellulose (HPMC) as a dissolution rate-enhancing polymer (Kerc, J., Srcic, S., Kofler, B., Farm. Vestn. 48, 284 (1997); Sugimoto, M., Okagaki, T., Narisawa, S., Koida, Y., Nakajima, K., Int. J. Pharm. 160, 11 (1998)).

Friedrich, et al (Friedrich H, Nada A, Bodmeier R. Drug Dev Ind Pharm. Sep; 31 (8): 719 (2005) provide an extensive review of the use of hydrophilic polymers in solubility enhancement. Many water soluble excipients were employed as carriers of solid solutions/dispersions. Among them, polyethylene glycols (PEG, Mw 1500-20000) were the most commonly used due to their good solubility in water and in many organic solvents, low melting points (under 65° C.), ability to solubilize some compounds and improvement of compound wettability.

The marketed Gris-PEG is the solid dispersion of griseofulvin in PEG 8000. The other carriers include PVP, polyvinylalcohol (PVA), polyvinylpyrrolidone polyvinylacetate copolymer (PVP-PVA), HPMC, hydroxypropyl cellulose (HPC), urea, Poloxamer 407, sugars, emulsifiers (SDS, Tween 80) and organic acids (succinic acid and citric acid). Because of the rapid dissolution of the water-soluble carriers than the drugs, drug-rich layers were formed over the surfaces of dissolving plugs, which prevented further dissolution of drug from solid dispersions. Therefore, surface-active or self-emulsifying agents including bile salts, lecithin, lipid mixtures, Gelucire 44/14 (Hülsmann et al., 2000) and Vitamin E TPGS NF (Khoo et al., 2000) were used as additional additives, acting as dispersing or emulsifying carriers for the liberated drug to prevent the formation of any water-insoluble surface layer. In addition, the release behaviors of many drugs are also improved by using water-insoluble polymers such as crospovidone (Hirasawa et al., 2003; 2004) and enteric polymers such as hydroxypropyl methylcellulose phthalate (HPMCP), cellulose acetate phthalate (CAP), Eudragit® L100 and S100 (Takada et al., 1989) and Eudragit® E (Horisawa et al, 2000; Jung et al., 1999).

Co-ground powders of the poorly water-soluble drug nifedipine and a hydrophilic carrier, [partially hydrolyzed gelatin (PHG), polyvinylpyrrolidone (PVP), sodium dodecyl sulfate (SDS), hydroxypropyl methylcellulose (HPMC), polyethylene glycol (PEG), urea or Pluronic F108] were prepared in order to improve the dissolution rate of nifedipine. The effects of type of grinding equipment, grinding time, and type of hydrophilic carrier on the crystallinity of nifedipine (x-ray diffraction and differential scanning calorimetry) on the interaction between drug and carriers (differential scanning calorimetry), on the particle size and appearance (scanning electron microscopy), on the wettability (contact angle measurements), and on the drug release were investigated.

Grinding nifedipine together with these carriers improved the dissolution rate. PHG-ground mixtures resulted in the fastest dissolution rate followed by PVP, SDS, HPMC, Pluronic, urea, and PEG. This effect was not only due to particle size reduction, which increased in the order PHG<PEG=SDS<Pluronic<drug<urea<HPMC<PVP, but also resulted from the ability of some carriers (PVP and HPMC) to prevent reaggregation of the finely divided drug particles. PVP, HPMC, and PHG formed a powder with amorphous drug.

The carriers improved the wettability of the ground products in the order HPMC<drug<urea<PVP<SDS<PHG<PEG<Pluronic. Differential scanning calorimetry (DSC) measurements gave valuable information about the nature of drug crystallinity and the interactions with the carriers within the ground mixtures.

Thus there is a need for a stable compositions of orlistat that can be taken up for down stream work for its conversion into a dosage form fro which the dissolution of the active ingredient is adequate. The prior art describes compositions that contain microcrystalline cellulose and polyvinylpyrrolidone as excipients; and are prepared by using an extruder.

Other avenues that can be employed, involve use of water soluble polymers as carriers or for preparation of molecular dispersions. However use of water soluble polymers as carriers or as a medium of a molecular dispersion entails application of techniques in which material would be subjected to elevated temperatures. Because of the low melting point of orlistat, it is obvious that such techniques would not be of much use.

### OBJECTS OF THE INVENTION

An object of the invention is to provide a composition comprising orlistat, which does not contain excipients like microcrystalline cellulose or polyvinylpyrrolidone and yet have adequate handling properties to enable it to be converted into a stable finished formulation.

Another object is to provide a process for preparation of composition comprising dispersion blend of orlistat and water soluble polymers as carriers and using a fluid bed processor.

### SUMMARY OF THE INVENTION

According to one aspect of the present invention there is provided a stable pharmaceutical composition comprising dispersion blend comprising 20 to 60% by weight of orlistat and 20% to 80% by weight of water soluble polymer carrier selected from hydroxypropyl methyl cellulose, methylcellulose, hydroxyethyl cellulose, hydroxypropyl cellulose or hydroxypropyl methylcellulose and the like.

### DETAILED DESCRIPTION OF THE INVENTION

Surprisingly, the inventors have found that particles containing orlistat and a water soluble polymer as a carrier in defined amount minimize the sticking and picking phenomena encountered during tablet compression or encapsulation and provide required dissolution properties.

The composition of the present invention is stable, does not stick during handling or become oily when exposed to temperature of 45-50° C., has optimum flowability and density for down stream work and exhibits necessary dispersibility and dissolution characteristics

The subject invention provides particles, such as granules and pellets, useful in producing pharmaceutical compositions, such as a unit dosage form. The use of particles in the form of granules is preferred.

In one of its aspects, the present invention relates to a composition comprising of a granular material which can be obtained in varying size range to suit the selected dosage form, wherein each particle comprises orlistat, a water soluble polymer as a carrier, a pharmaceutically acceptable surfactant and an anti-tack material, such as, talc or silicone dioxide. It was also found that it is not critical to employ microcrystalline cellulose to make an acceptable formulation and an acceptable formulation can be made without using an extruder.

The water soluble polymer are selected from cellulose ethers like HPMC, hydoxypropyl cellulose and ethyl cellulose.

Preferred pellets additionally contain from about 1% to about 2% by weight polysorbate 80, 0.5% to 2% pregelatinized starch as a disintegrant and about 0.5% talc or silicone dioxide as anti-tack.

Such products are chemically stable and can be filled on fast running encapsulation machines without presenting the sticking and picking phenomena

Granules or pellets are preferably prepared by using a fluid-bed processor.

It has been found that the water soluble, modified or substituted celluloses, for example methylcellulose, hydroxyethyl cellulose, hydroxypropyl cellulose or hydroxypropyl methylcellulose give a stable product that affords prompt dissolution of orlistat.

It is also found that the composition prepared by the method using the fluid bed processor provides a material that can be conveniently subjected to common down stream pharmaceutical manipulations like, size reduction, sizing, blending, encapsulation in hard gelatin capsules and tableting.

It was also found that the compositions of this invention do not ooze molten orlistat when exposed to a temperature of 50° C. They become soft due to melting of orlistat. However, as the temperature is brought down to below 30° C., they regain their original consistency and can be conveniently taken up for down stream work. The dissolution of orlistat also is not affected by the effect of elevated temperature.

The drug-carrier combination can be prepared through coprecipitation of the two or more compounds from a common solvent. They can also be prepared by precipitation of the water insoluble drug suspended in a solution of the carrier. The present invention is about a using a fluid bed processor to accomplish coprecipitation of the drug-polymer-surfactant coprecipitate from a common solvent on to a pharmaceutically acceptable excipient or excipient mixture. The approach involves a fluidized bed coating system, wherein a drug suspension in the carrier solution is sprayed onto the granular surface of a suitable excipient to produce either granules ready for tableting or drug-coated granules for encapsulation in one step.

The process can be carried out in a fluid bed processor using a bottom spray, a top spray or a tangential spray attachment. The flowability, processability and other characteristics of the drug-carrier combination of the invention can be readily controlled through the choice of appropriate pharmaceutically acceptable excipients onto which the drug-polymer solution is coated; and by varying the process variables like the spray rate and the degree of fluidization. Further control on the particle size distribution can be exercised by subjecting the molecular dispersion obtained from the process to a size-reduction step.

The flowability of orlistat is significantly enhanced by its conversion into the molecular dispersion blend as described herein. Such a significant improvement in the flow properties of a material indicates superior handling capabilities during processing into pharmaceutical dosage forms.

The preferred embodiment of this invention involves a process comprising (a) preparing a solution of a water soluble polymer along with a surfactant in water, (b) dispersing finely powdered orlistat in this solution, and (c) spraying this solution onto a substrate consisting of a pharmaceutically acceptable excipient in a fluid bed processor. The particle thus obtained are subjected to down stream techniques and converted in finished dosage forms, like capsules and tablets.

The following Examples are illustrative but in no way limit the invention.

### EXAMPLE 1

Various proportions of the selected excipients were evaluated to arrive at the optimum formulation from the point of view of physical stability and dissolution. The details are given below:

TABLE
Effect of HPMC concentration on properties of the composition*

| | Composition No. | | | |
|---|---|---|---|---|
| | 1 | 2 | 3 | 4 |
| HPMC | 10 | 15 | 20 | 30 |
| Concentration (%) | | | | |
| Flow properties | Sticky, waxy | Sticky, waxy mass, | Free flowing | Free flowing |
| | mass | Flow not adequate | powder | powder |
| | Does not flow | | | |
| Appearance when | Melts and | Melts to a | Becomes soft | Becomes soft |
| exposed | acquires a | gel like mass | but regains its | but regains its |
| To 45° C. | liquid like | | appearance | appearance |
| | appearance | | when cooled | when cooled |
| Dissolution(% | 68 | 75 | 88 | 91 |
| dissolved in 45 | | | | |
| minutes**) | | | | |

*The concentration of the active ingredient at 50% and that of polysorbate 80 at 2% was kept constant. The quantity of sugar granules/pellets was varied depending on the HPMC concentration used.
**Apparatus: Paddle, 100 rpm
Medium: 0.1 N hydrochloric acid with 2% sodium lauryl sulphate.

This shows that at concentration of or above 20% HPMC provides free flowing and stability properties as well the dissolution much better than when present in lesser amount. This demonstrates that in the given amount HPMC acts as carrier to provide required properties to the composition.

### EXAMPLE 2

Preparation of Pellets

a) 45 g polysorbate 80 and 450 g hydroxypropylmethyl cellulose are dissolved in sufficient quantity of demineralized water by means of a stirrer.

b) 1150 g orlistat is suspended in the solution prepared in step 1

c) 750 g of sugar granules or pellets are coated with the orlistat suspension made in step 2 at a temperature not exceeding 35° C. in bottom-spray attachment of a fluid bed processor.

d) After complete quantity of the orlistat suspension is consumed, the material is dried in a fluid bed or a tray drier equipped with a dry air supply at a temperature not exceeding 35° C. to a moisture content of less than 2.5%.

e) The dried material is sized in the manner described in Example 1, mixed with 2.5 g of fumed silica and collected in tightly packed containers.

f) The composition thus made is tested for dissolution of orlistat using a paddle type of dissolution test apparatus at 100 rpm and 0.1 N hydrochloric acid containing 2% w/w of sodium lauryl sulphate as the medium. The material is also subjected to slightly elevated temperatures to study the effect of heat on the appearance, handling properties and the dissolution.

### EXAMPLE 3

Tablets containing 120 mg of orlistat were prepared from the following:

Manufacturing process:

a. About 240 g of orlistat granules prepared in Example 1 are blended with 20 g pregelatinized starch, 6 g of croscarmellose sodium, 2 g of fumed silica and 2.5 g of magnesium stearate.

b. The blend is compressed into tablets using a round, biconvex tooling of 9 mm size.

c. The composition thus made is tested for dissolution of orlistat using a paddle type of dissolution test apparatus at 100 rpm and 0.1 N hydrochloric acid containing 2% w/w of sodium lauryl sulphate as the medium. The material is also subjected to slightly elevated temperatures to study the effect of heat on the appearance, handling properties and the dissolution.

The results are provided in Example 4.

### EXAMPLE 4

The composition of orlistat described in the above examples were tested for dissolution of orlistat using a paddle type of dissolution test apparatus at 100 rpm and 0.1 N hydrochloric acid containing 2% w/w of sodium lauryl sulphate as the medium. The samples for the testing were filled in hard gelatin capsules and their dissolution was compared with Xenical®, the innovator's marketed formulation). The results are given below—

| | % orlistat dissolved |
|---|---|
| Composition | in 45 minutes |
| Example 2 | 82.4 |
| Example 3 | 81.3 |
| Xenical(RTM) | 80.2 |

This demonstrates that compositions of orlistat with adequate dissolution properties could be made without using microcrystalline cellulose. Microcrystalline cellulose is considered mandatory in the innovator's compositions.

### EXAMPLE 5

The affect of elevated temperature on appearance and handling properties on the orlistat compositions of this invention was studied by slowly increasing the temperature of a hot air oven in which these compositions were kept in Petri dishes. Pure orlistat also was subjected to this study. To carry out a comparison with the innovator's formulation Xenical®, its contents were emptied in a Petri dish. The observations of this study are given below—

| Temperature | | | Xenical | |
|---|---|---|---|---|
| ° C. | Example 2 | Example 3 | (RTM) | Orlistat |
| 30 | No change | No change | No change | No change |
| 35 | -do- | -do- | -do- | -do- |
| 37 | -do- | -do- | -do- | softens |
| 40 | No change | NO change | No change | Starts melting |
| 45 | Slightly soft and | Slightly soft | Slightly soft and | Melts |
| | sticky | | sticky | completely |
| 50 | -do- | -do- | Slightly soft and | -do- |
| | | | sticky, becomes | |
| | | | powdery on | |
| | | | touching | |
| Cooled down | Regains its | Regains its | Regains its | Does not retain |
| to less than | original | original | original | its original |
| 30° C. | hardness, | hardness, | hardness, | appearance |
| | dissolution is not | dissolution is not | dissolution is not | |
| | affected | affected | affected | |

Thus the composition of present invention leads to composition which is comparable to the innovator in terms of appearance and handling properties at elevated temperatures even without use of stabilizers like microcrystalline cellulose.

1. A stable pharmaceutical composition comprising dispersion blend comprising 20 to 60% by weight of orlistat and 20% to 80% by weight of water soluble polymer carrier selected from hydroxypropyl methyl cellulose, methylcellulose, hydroxyethyl cellulose, hydroxypropyl cellulose, hydroxypropyl methylcellulose and the like.

2. Composition as claimed in claim 1 wherein water soluble polymer is hydroxypropyl methyl cellulose.

3. Composition as claimed in claimed in claim 1 additionally comprising polysorbate 80, pregelatinized starch as a disintegrant and talc or silicone dioxide as anti-tack.

4. Composition as claimed in claim 3 wherein polysorbate 80 is present in amount of 1% to about 2% by weight.

5. Composition as claimed in claim 3 wherein pregelatinized starch is present in amount of 0.5% to 2%.

6. Composition as claimed in claim 3 wherein talc or silicone dioxide 0.5% as anti-tack.

7. The composition as claimed in claim 1 is in the form selected from granules, pellets, preferably pellets.
